(19) United States
(12) Patent Application Publication
(10) Pub. No.: US 2008/0086075 A1
(43) Pub. Date: Apr. 10, 2008

(54) VASCULAR ACCESS DEVICES AND METHODS OF USE

(76) Inventors: F. Frank Isik, Mercer Island, WA (US); Thomas Robert Kirkman, Craig, AK (US)

Correspondence Address: LEVINE BAGADE HAN LLP, 2483 EAST BAYSHORE ROAD, SUITE 100, PALO ALTO, CA, 94303, US

(21) Appl. No.: 11/864,446

(22) Filed: Sep. 28, 2007

Related U.S. Application Data
(60) Provisional application No. 60/828,746, filed on Oct. 9, 2006.

(51) Int. Cl. A61M 39/06

(52) U.S. Cl. 604/27

(57) ABSTRACT

Vascular access devices and methods of use may utilize a single access port which is attached to a vessel wall and allows for controlled insertion of small to large sized instruments and catheters. The access port may be secured via securement mechanisms deployed from within the vessel lumen. Also, one or more cutting blades may be utilized to create and/or define the individual flaps in the access port and/or underlying tissue wall at the time of port deployment and securement. Such an access port allows a user to access and/or re-access the same artery and/or vein of patients utilizing various diameter catheters and instruments while maintaining hemostasis.

### CROSS-REFERENCE TO RELATED APPLICATIONS

This application claims the benefit of priority to U.S. Prov. Pat. App. 60/828,746 filed Oct. 9, 2006, which is incorporated herein by reference in its entirety.

### FIELD OF THE INVENTION

The present invention relates to devices and methods for accessing and/or controlling vascular access puncture sites. More particularly, the present invention relates to devices and methods for accessing and/or controlling entry through vascular puncture sites via self-adjusting entry devices.

### BACKGROUND OF THE INVENTION

The increasing success of interventional techniques to access and repair structural disorders of the heart and vascular system has led to increasing demand for such procedures. Methods to deploy either intra-vascular stents or valve repair devices generally utilize the insertion of catheters through arteries and veins in the upper or lower extremities. As the technology and the ability to treat a wider-range of medical conditions evolve, the devices delivered have increased in size. Accordingly, closure of larger sized holes left by larger diameter catheters may be problematic for a patient.

A common cause of patient morbidity for interventional techniques is vascular access site complications, such as hematomas, pseudoaneurysms, and retroperitoneal bleeding. Such complications are likely to increase in frequency and severity with anti-coagulation and the use of larger diameter catheters used to deliver the endovascular devices. These complications may lead to prolonged hospital stay, increased costs, and the possible need for transfusion or surgery. Additionally, complications may lead to patient dissatisfaction and discomfort.

Manual compression of a vascular access site is typically utilized to achieve hemostasis of the opening when the size of the catheter sheath used is 6 F or less. But endovascular treatment of larger aneurysms and valular diseases in an anti-coagulated patient generally require catheter sheaths in the range of 18-24 F. Although a cut-down can be performed by a vascular surgeon to directly close the access site in the artery or vein, alternative and less invasive methods are desirable. Furthermore, dilation of the artery or vein by the increasing diameter catheters can lead to damage and tearing of the vessel wall, making them less amenable to direct closure.

Conventional methods and devices used to close vessel puncture sites or ports, typically in the 6-8 F range, generally fall into the following categories: direct pressure, sealant-based devices, suture-based devices, staple-based devices, and direct closure by cut-down and vascular suture. However, each of these methods and devices has their limitations. For instance, most of these methods and devices have failure rates of up to 30% when utilized on relatively large diameter holes, e.g., 18 F or greater. Moreover, suture or staple-mediated devices also have the disadvantage of potentially narrowing the artery caliber and thus are contraindicated for use in relatively small vessels, e.g., 5 mm or less. Additionally, procedures requiring repeated access to vessels may require the creation or multiple access sites as closure by many conventional devices and methods fail to allow for repeated access through the same site.

Accordingly, there is a need for methods and devices which allow for the controlled access by any number of various sized devices to any number of various diameter vessels while maintaining homeostasis as well as for allowing repeated access to a vessel through a single access site as necessary or desirable.

### SUMMARY OF THE INVENTION

Access ports and methods of use for controlling access to vascular bodies may allow for a single access port which is adhered, connected, or otherwise attached to a vessel wall and allows for, but is not limited to, control of small to large sized vascular defects, use with anticoagulation agents, rapid sheath removal, early ambulation of the patient, access through the same port, maintaining a size of the vessel lumen after repair etc. Moreover, such an access port may allow a user to access and/or re-access the same artery and/or vein of patients utilizing various diameter catheters and instruments.

When an instrument or catheter is inserted through the flaps of such an access port, the flaps may be pushed inwardly into the vessel lumen to provide a channel for passage of the instrument or catheter sheath while the access poll shields the vessel wall from damage. The outer periphery of the access port may remain intact and the flaps may allow the insertion of various sized catheter sheaths. Removal of the instrument or catheter may allow for the return of the patch flaps to a neutral position. The access port would allow re-access of the vessel, if necessary, even in the anti-coagulated patient.

Generally, the access port may comprise a flexible patch sized for securement upon a vessel lumen, where the patch may define an opening therethrough with one or more flaps, at least one elastically deformable scaffold member integrated with the patch, wherein the one or more flaps are deformable into a open port configuration when an instrument or catheter is inserted through the opening, and where the at least one member is further biased to reconfigure the one or more flaps back to a closed port configuration upon removal of the instrument or catheter.

The access port may be secured via one or more securement mechanisms deployed optionally from within the vessel lumen. Additionally, one or more cutting blades may be utilized to create and/or define the individual flaps in the access port and/or underlying tissue wall at the time of port deployment and securement.

When deployed and in use, one exemplary method for securing the access port to the vessel lumen may generally comprise advancing a piercing and securement assembly in a low profile configuration through the vessel opening into the vessel lumen, expanding the piercing and securement assembly into a deployment configuration within the vessel lumen, compressing tissue surrounding the vessel opening between the piercing and securement assembly and a distal end of a housing shaft such that the access port is secured to an outer surface of the vessel lumen, reconfiguring the piercing and securement assembly into its low profile configuration, and withdrawing the piercing and securement assembly from the vessel lumen through the access port such that one or more flaps defined on the vascular port are configured from an open port configuration to a closed port configuration upon withdrawal.

### BRIEF DESCRIPTION OF THE DRAWINGS

FIG. 1 illustrates one variation of a device with a piercing and securement assembly positioned through a vessel wall prior to securement of a deployable access port.

FIG. 2A illustrates a detail partial cross-sectional view of a piercing and securement assembly positioned within a vessel with one or more securement mechanisms to be mated against the access port.

FIG. 2B illustrates the device of FIG. 2A utilizing a guidewire passed through a lumen defined through the device to facilitate access into the vessel.

FIGS. 2C to 2G illustrate a method for confirming suitable placement or positioning within a vessel prior to deployment of the access port.

FIGS. 3A to 3G illustrate one method where a piercing and securement assembly is pierced through a vessel wall in a low profile configuration and then expanded into an expanded or deployment configuration for securing an access port to a surface of the vessel.

FIGS. 4A and 4B illustrate one variation for deploying or reconfiguring a piercing and securement assembly from its low profile configuration into an expanded or deployment configuration utilizing an expansion mechanism.

FIGS. 5A and 5B illustrate top views of one variation of an access port prior to deployment and having one or more flaps created through the port for deployment, respectively.

FIGS. 6A and 6B show side views of an access port secured along a vessel surface with one or more catheter shafts being advanceable into or withdrawn from the access port.

FIG. 7 illustrates a side view and partial cross-sectional detail view of another variation of an access port secured along a vessel wall and having overlapping flap surfaces to facilitate hemostasis through the access port.

FIG. 8 shows a top view and partial cross-sectional side view of another variation of an access port having interlockable or interfittable flap surfaces.

FIGS. 9A and 9B show top views of yet another variation of an access port having at least three flaps illustrating closure of the flaps relative to one another upon withdrawal of a shaft from between the flaps.

FIGS. 10A and 10B show top views of yet another variation of an access port having one or more sutures passed between adjacent flaps for optionally securing the flaps via the sutures.

FIGS. 11A and 11B illustrate perspective and cross-sectional side views, respectively, of yet another variation of an access port having integrated deployable members for securing the access port to a vessel.

### DETAILED DESCRIPTION OF THE INVENTION

Vascular access control devices and methods of use may allow for a single access pole which is adhered, connected, or otherwise attached to a vessel wall and allows for, but is not limited to, control of small to large sized vascular defects (e.g., large sized vascular defects or openings may range anywhere from 12 F-24 F), use with anticoagulation agents, rapid sheath removal, early ambulation of the patient, access through the same port, maintaining a size of the vessel lumen after repair, etc.

Moreover, such an access port may allow a user to access and/or re-access the same artery and/or vein of patients utilizing various diameter catheters and instruments. For instance, patients who may require long-term indwelling catheters or those who require repeated intravascular access, e.g., hemodialysis patients, may benefit. Thus, after a procedure utilizing the access port, the tissue region surrounding the access port may be closed upon the port and left implanted in the patient as the access port provides hemostasis of the vessel. If re-entry or further access is desired to the vessel for any further procedures, the access port may again be entered through the tissue region for the re-entry or re-introduction of one or more instruments or catheters through the same port without having to create any additional entry paths into the vessel lumen. The re-entry or further access through the access port may be obtained intra-operatively or post-operatively spanning anywhere from hours, days, weeks, months, or even years from an initial procedure. Moreover, the access port may be left implanted within the patient permanently, if so desired, to provide this re-entry path into the vessel for future use or it may be optionally removed at any time and the opening through the vessel may be closed.

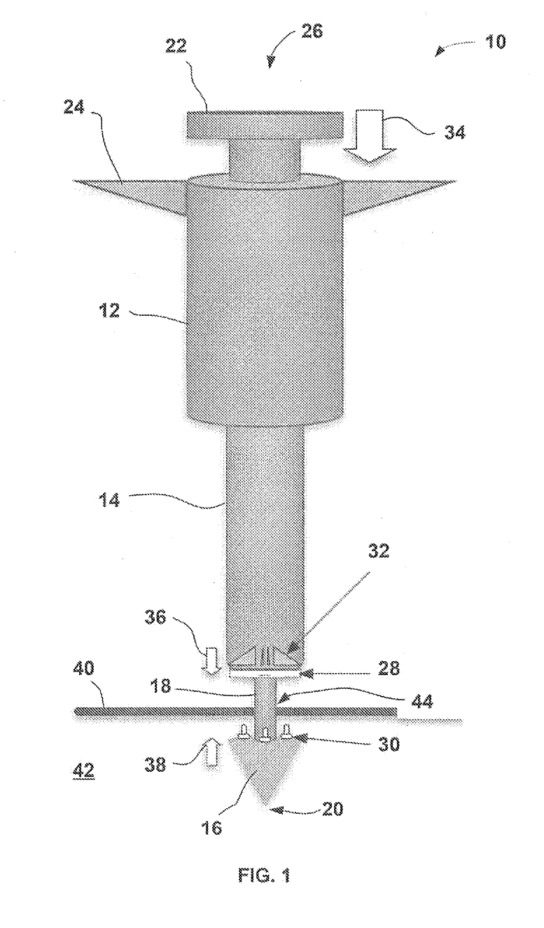

Turning to FIG. 1, an example of such a device is illustrated in vessel entry assembly 10. Vessel entry assembly 10 may allow a user to create a controlled vascular opening by maintaining a vascular puncture while protecting the vessel during insertion and/or withdrawal of relatively large diameter sheaths, catheters, or instruments. Assembly 10 may deploy and secure a deployable access port 28 within or against the vessel wall where the access port 28 may allow for the automatic closure and sealing of a vascular opening to maintain hemostasis when instruments or catheters are withdrawn from the port 28, as described in further detail below.

As shown, vessel entry assembly 10 may generally comprise a body or housing 12 which may be held by the user during a procedure. A housing shaft 14 may extend distally from body 12 and an additional piercing and securement assembly 16 connected to elongate shaft 18 may be translatably actuated relative to body 12, as described below in further detail.

Piercing and securement assembly 16 may be tapered into a piercing tip 20 to facilitate entry through a vessel wall 40 and access into the vessel lumen 42. Plunger 22 may positioned along a proximal end of body 12 to provide counterforce relative to handles 24 projecting from body 12 when assembly 10 is actuated for securing access port 28 to the vessel wall 40. A central lumen 26 may be optionally defined through the assembly 10 through which any number of instruments or agents, such as contrast agent, may be passed through. In one variation, a guidewire may be passed through lumen 26 to facilitate entry into the vessel lumen 42, such as used during entry methods such as the Seldinger technique.

Generally in use, piercing and securement assembly 16 may be configured into a low profile shape for initially piercing through vessel wall 40 and creating vessel opening 44 into vessel lumen 42. In the variation utilizing a central lumen 26 through assembly 10, contrast may be injected through the device and into the vessel 26 to confirm appropriate positioning of piercing and securement assembly 16 within lumen 42. Moreover, a guidewire may be passed through assembly 10 and into vessel lumen 42 to facilitate entry and access into the vessel 40.

Once within lumen 42, piercing and securement assembly 16 may be expanded or reconfigured into its deployed profile, as shown in FIG. 1, to reveal one or more securement mechanisms 30 along a proximal portion of piercing and securement assembly 16. Securement mechanisms 30 may comprise any number of securement members such as rivets, screws, projections, keyed members, magnetic elements, etc. which are removably held along piercing and securement assembly 16 in apposition to the interior of vessel wall 40 and access port 28.

Access port 28 may have one or more openings or receiving channels (or complementary magnets having an opposite polarity to those members 30 held by piercing and securement assembly 16), which correspond in alignment and in number with securement mechanisms 30. When piercing and securement assembly 16 and securement mechanisms 30 are urged proximally into contact against the interior of vessel wall 40, the portion of vessel wall 40 may become compressed between assembly 16 and access port 28 positioned near or at a distal end of housing shaft 14 such that securement mechanisms 30 may at least partially pierce through vessel wall 40 and into receiving contact with the corresponding openings on access port 28. Moreover, any number of securement mechanisms 30 may be utilized provided that the number and placement of mechanisms 30 against access port 28 is sufficient to secure the access port 28 against or to the vessel wall 40. For instance, four or more securement mechanisms 30 may be uniformly spaced such that they secure against access port 28 uniformly against the tissue.

Optionally, one or more actuatable cutting blades 32 may be positioned within the distal end of housing shaft 14 proximally of access port 28. For instance, four or more cutting blades 32 may be uniformly spaced within shaft 14 such that when handles 24 and/or plunger 22 are distally urged 34 to thereby distally urge 36 housing shaft 14, the cutting blades 32 may cut through a portion of access port 28 and at least partially through vessel wall 40 to create two or more retractable flaps, is described in further detail below. Alternatively, housing shaft 14 may be urged distally while assembly 16 and securement mechanisms 30 are urged proximally 38 in a simultaneous motion to bring securement mechanisms 30 into contact with access port 28.

The access port 28 may be appropriately sized to access a variety of catheter or instrument diameters, e.g., catheter sheaths ranging in size anywhere from 12 F-24 F. Moreover, assembly 10 and/or access port 28 may be designed to be disposable after use for a single-use application.

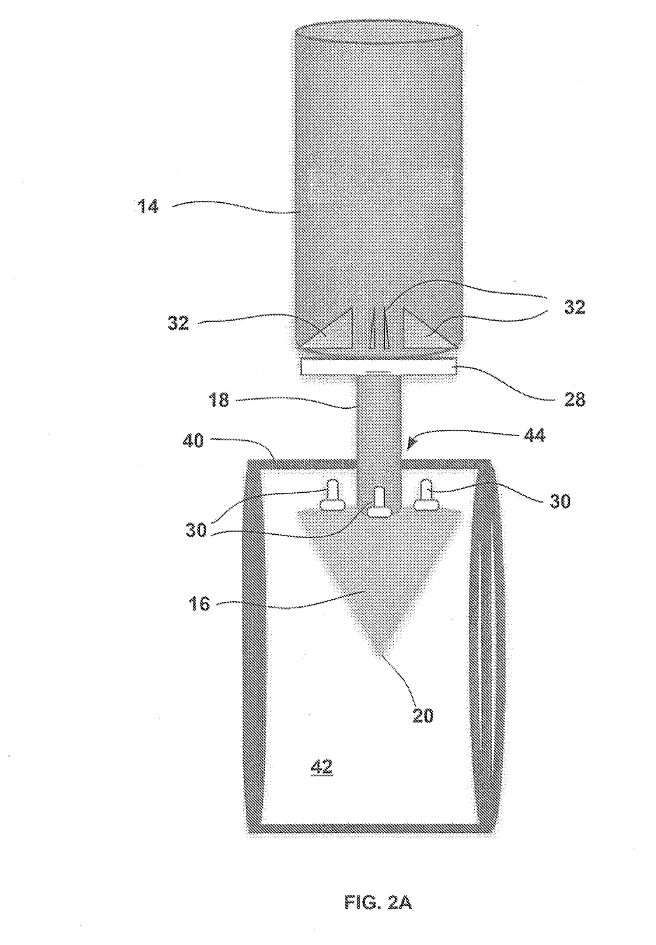

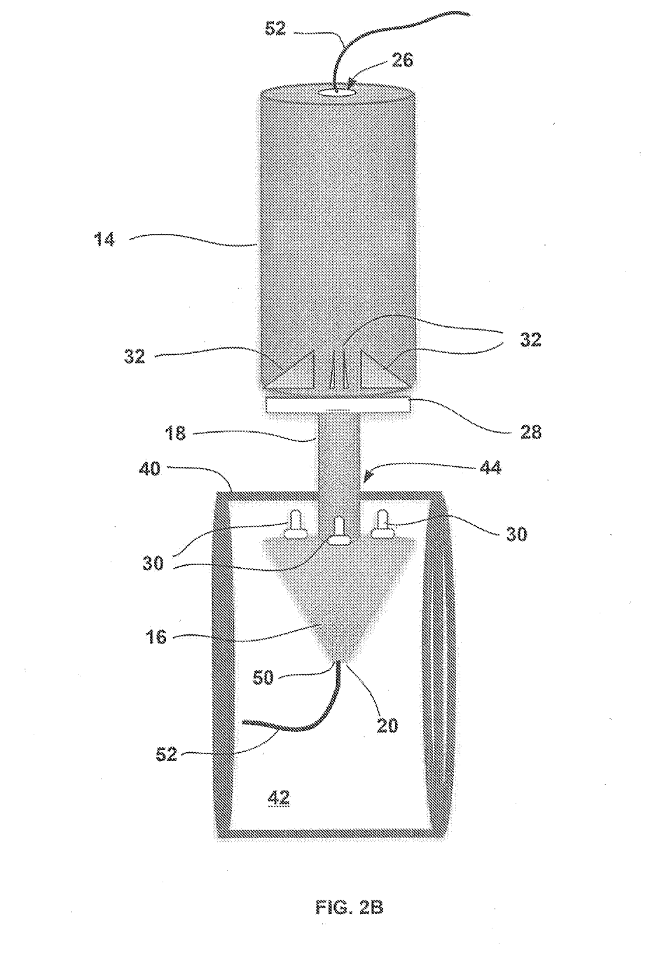

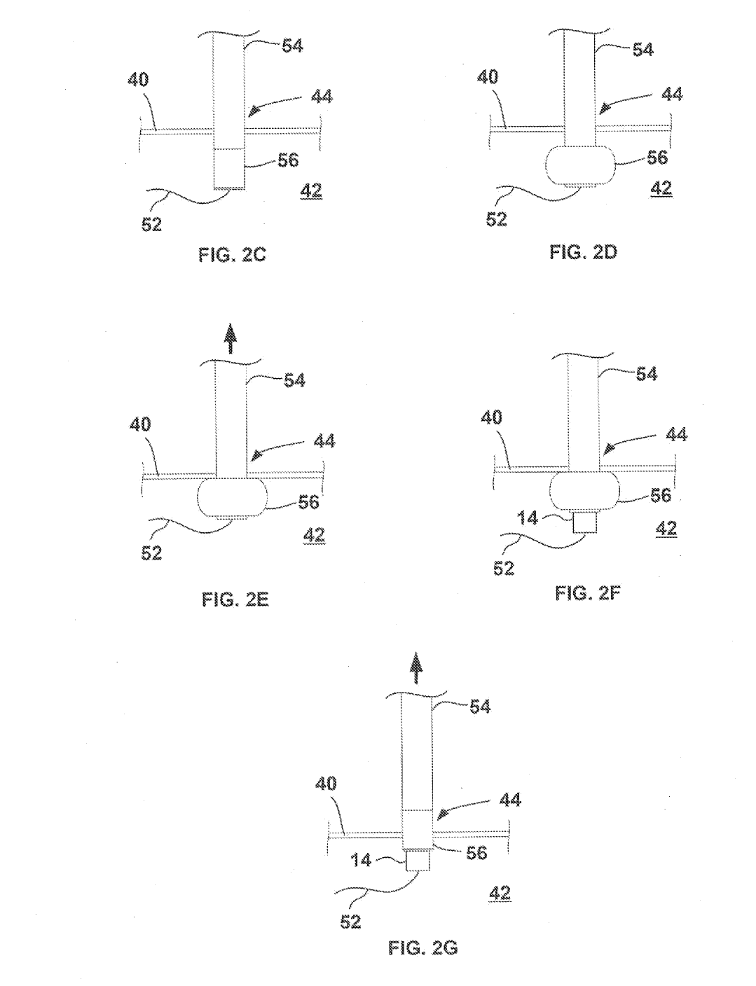

FIG. 2A shows a partial cross-sectional detail view of piercing and securement assembly 16 positioned within vessel lumen 42 in its deployed configuration with multiple securement mechanisms 30 positioned in apposition against vessel wall 40 for engagement with access port 28. Also illustrated are cutting blades 32 for cutting into access port 28 and/or vessel wall 40 for creating the two or more retractable flaps.

FIG. 2B illustrates a partial cross-sectional detail view of an alternative variation of a device having central lumen 26 defined through assembly 10. As shown, guidewire 52 may be passed through central lumen 26 such that its exits through guidewire lumen opening 50 and into vessel lumen 42. Guidewire 52 may be optionally maintained through vessel opening 44 even when assembly 10 is withdrawn from the vessel wall 40 to facilitate the entry and guidance of additional instruments.

In yet another variation for confirming suitable placement or positioning within a vessel prior to deployment of the access port 28, an outer sheath or catheter 54 may introduced through vessel opening 44 over or along guidewire 52, as shown in FIG. 2C. Catheter 54 my have an inflatable balloon or member 56 positioned near or at a distal end of catheter 54 which is introduced through opening 44 in its deflated configuration but once placed through opening 44 and within vessel lumen 42, balloon 56 may be inflated via an inflation lumen defined through catheter 54 to a diameter which is greater than a diameter of vessel opening 44, as shown in FIG. 2D. Although balloon or member 56 is illustrated as a balloon, it may be configured alternatively as an expandable scaffold or one or more radially extendable members.

With balloon or member 56 inflated or expanded, catheter 54 may be retracted proximally such that balloon 56 is pulled against the interior surface of the vessel 40 until resistance is felt by the physician, as shown in FIG. 2E. The absence of any resistance may indicate that the catheter 54 or balloon 56 is improperly positioned with respect to the targeted vessel 40 and that reintroduction or repositioning may be desirable. Once the positioning through vessel opening 44 has been determined as being suitable for deploying the access port 28, housing shaft 14 may be advanced through catheter 54 along guidewire 52 until entry into vessel lumen 42 has been achieved, as shown in FIG. 2F.

Although housing shaft 14 is illustrated as being introduced through a lumen within catheter 54, other variations may include advancing housing shaft 14 over or along catheter 54 such that a distal end of housing shaft 14 is advanced into apposition against an exterior of vessel 40. In either case, once housing shaft 14 has been suitably positioned with respect to the targeted vessel 40, balloon or member 56 may be deflated and withdrawn proximally from vessel lumen 42, as shown in FIG. 2G, to allow for the deployment and securement of access poll 28, as described herein.

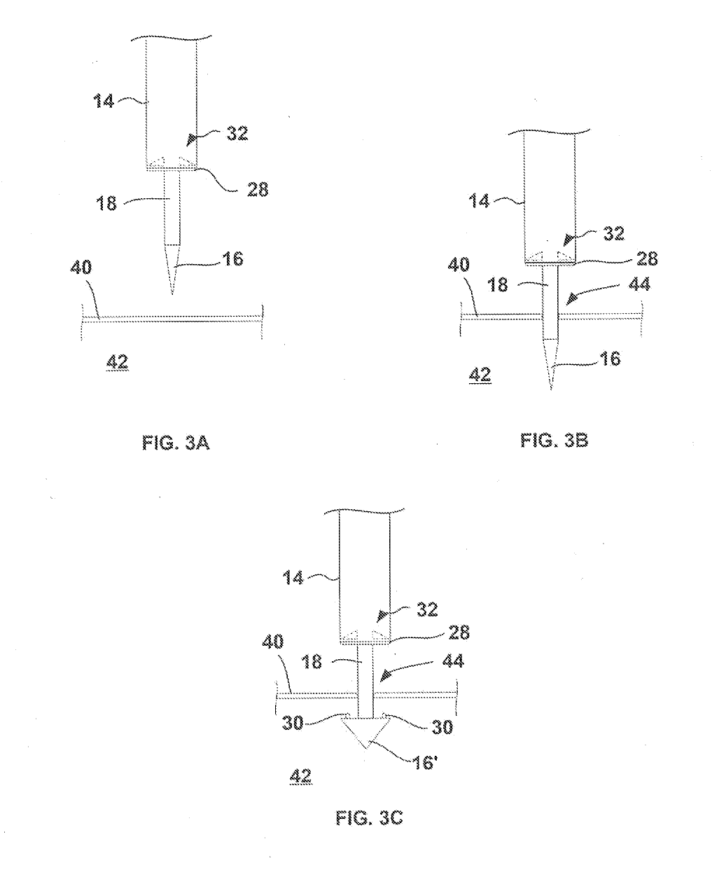

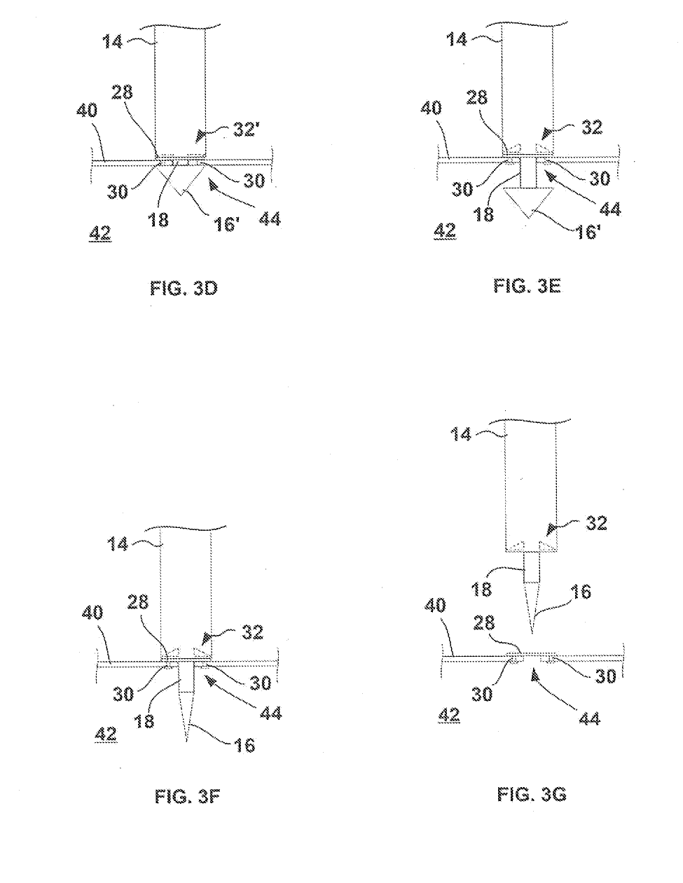

FIGS. 3A to 3G illustrate one method for creating the initial access and deployment of access port 28 against or upon vessel wall 40. As shown in FIG. 3A, elongate shaft 18 and piercing and securement assembly 16 may be pierced into the patient body, e.g., at a femoral access point, to approach vessel wall 40 while piercing and securement assembly 16 is in a low profile configuration. Piercing and securement assembly 16 may be advanced through vessel wall 40 to create vessel opening 44, as shown in FIG. 3B, until piercing and securement assembly 16 is at least partially within vessel lumen 42. Any number of optional indicators for detecting appropriate entry of the device into vessel lumen 42 may also be incorporated. For instance, a portion of the shaft 18 or central lumen 26 may be fabricated from a clear plastic or glass to receive any flash of blood entering from vessel lumen 42 as a visual indication that the piercing and securement assembly 16 has appropriately entered into the vessel lumen 42.

Once desirably positioned within vessel lumen 42, piercing and securement assembly 16 may be actuated or reconfigured to expand into its deployed configuration 16′ while exposing and positioning the one or more securement mechanisms 30 into apposition relative to the interior of vessel wall 40 and access port 28, as shown in FIG. 3C. Access port 28, which is removably positioned near or at the distal end of housing shaft 14, and/or assembly 16′ may then be actuated towards one another to compress or sandwich the portion of vessel wall 40 around opening 44 between access port 28 and assembly 16′, as shown in FIG. 3D. Advancement or retraction of housing shaft 14 and/or elongate shaft 18 may be ratcheted or keyed to control the relative movement in a controlled manner.

As the tissue is compressed, securement mechanisms 30 are brought into piercing contact against the interior of vessel wall 40 until they are received or engaged in a securing manner by corresponding openings or channels defined on access port 28. Additionally, the one or more blades 32′ positioned within the distal end portion of housing shaft 14 may also be actuated to press against access port 28 to at least partially cut into or through port 28 to create the two or more retractable flaps. The blades 32′ may be further actuated to optionally cut into or through the tissue wall 40 surrounding vessel opening 44 to further create two or more flaps in the tissue beneath port 28 as well.

Once the securement mechanisms 30 have been engaged by port 28 to secure port 28 against or upon vessel wall 40 and once the optional cuts have been made by actuated blades 32′ in port 28 and/or the underlying tissue, blades 32 (if utilized) may be withdrawn from contacting port 28 and assembly 16′ may be advanced distally to release the tissue and also to release securement mechanisms 30 therefrom, as shown in FIG. 3E. With assembly 16′ disengaged from the tissue, assembly 16′ may be reconfigured from its expanded or deployed profile back into its low profile configuration 16, as shown in FIG. 3F. Then, elongate shill 18 and reconfigured assembly 16 may be withdrawn proximally from vessel opening 44 while disengaging and leaving access port 28 attached or adhered to vessel wall 40, as illustrated in FIG. 3G.

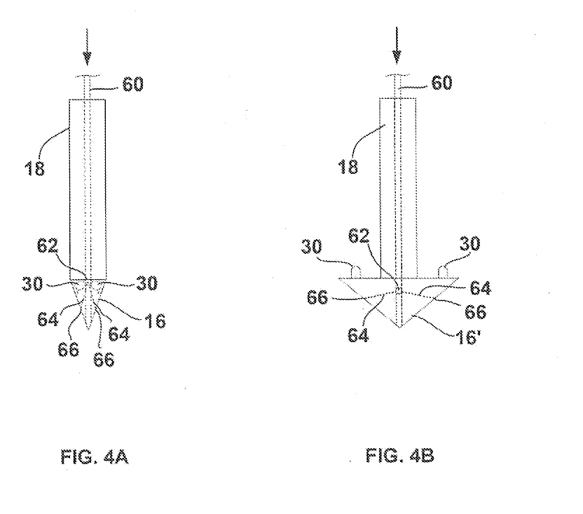

Although piercing and securement assembly 16 may be expanded or deployed from a low profile configuration utilizing any number of mechanisms, one example of such a mechanism is shown in the profile views of FIGS. 4A and 4B. As illustrated in FIG. 4A, assembly 16 may be maintained in its low profile configuration where its outer surface is contiguous with the outer surface of elongate shaft 18. The one or more securement mechanisms 30 may be positioned within assembly 16 such that they are completely contained within the device during delivery. An actuation shaft 60 may slidably extend through elongate shaft 18 and be coupled to sliding member 62, which is in turn pivotably attached to one or more support members 64. The one or more members 64 may in turn be coupled or pivotably attached at attachment points 66 to an interior of assembly 16.

In use, actuation shaft 60 may be pushed distally relative to elongate shaft 18 such that actuation shaft 60 in turn urges sliding member 62 distally thus forcing the one or more support members 64 to expand or deploy assembly 16′ into its opened configuration, as shown in FIG. 4B. As assembly 16′ is urged into its deployment configuration, securement mechanisms 30 may be exposed and repositioned into apposition with respect to housing shaft 14 and access port 28.

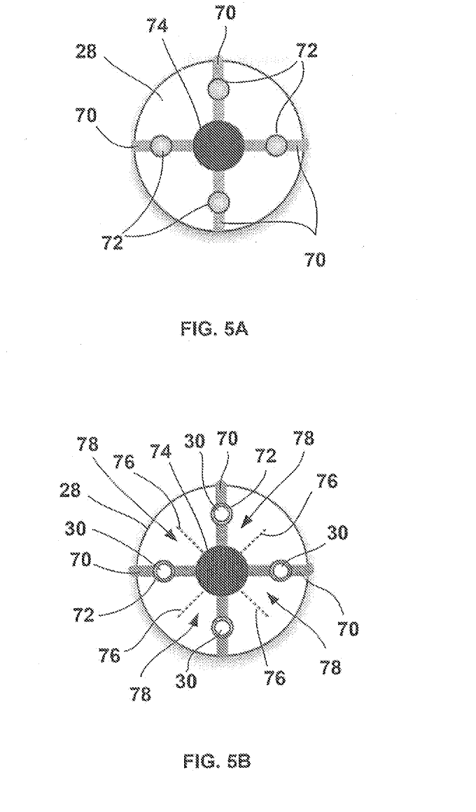

Turing now to the access port, the deployable port may be fabricated into a variety of shapes and configurations. For example, port 28 may be configured into a circular disk or discoid patch. Other shapes, including but not limited to, elliptical, rectangular, triangular, etc. shapes may be alternatively utilized depending upon the desired area to be accessed. One particular variation is shown in the top views of FIGS. 5A and 5B where access port 28 may be formed into a circularly shaped patch. Such an access port 28 may be fabricated from any variety of flexible biocompatible materials such as ePTFE, other fluoropolymers, polymers, polymeric blends thereof, elastomers, etc.

One or several frame or scaffold members 70 may be integrated within access port 28 to provide structural support and maintenance of a configuration of port 28. Scaffold members 70 may be positioned in various configurations to support access port 28, such as a crossed configuration as shown, a circular frame configuration, or any various shapes and configurations provided that a shape of access port 28 is sufficiently maintained during delivery and deployment in a patient body. Moreover, a portion or the entire scaffold members 70 may be fabricated from spring stainless steel, super-elastic alloys, or shape memory alloys such as Nickel-Titanium alloys, e.g., Nitinol. As shown, one or more receiving openings 72 may be located along scaffold members 70 for receiving the securement mechanisms 30 in a corresponding manner. A central opening 74 may be optionally defined on access port 28, which can be controlled for hemostasis at the conclusion of a procedure by application of direct pressure or which can be closed utilizing a variety of procedures, e.g., suturing, clips, adhesives, etc.

Moreover, any of access port 28 and/or scaffold members 70 and/or securement mechanisms 30 may be alternatively fabricated from any variety of biodegradable, bioerodable, or bioabsorbable materials, as desired.

Upon placement and securement of access port 28 upon or against the vessel wall 40, the securement mechanisms 30 may be introduced within corresponding receiving openings 72 to secure the port 28 to the tissue, as shown in FIG. 5B. Additionally, the blades 32 (if utilized) may be actuated to cut 76 at least partially through the access port 28 between scaffold members 70 to create at least one or more retractable flaps 78. With the access port 28 cut to create the flaps 78, the frame members 70 extending along the portion of the newly-created flaps 78 may function as retraction members to retract a flap 78 back to its un-biased shape when temporarily deformed by the insertion of an instrument or catheter through central opening 74.

Alternatively, access port 28 may incorporate expandable biomaterials along the seams between the flaps 78 to allow swelling and expansion and seal the seams when placed in contact with blood or when temperature is increased. In yet other alternatives, access port 28 may also incorporate drug-eluting agents to facilitate the healing of the acute wound site.

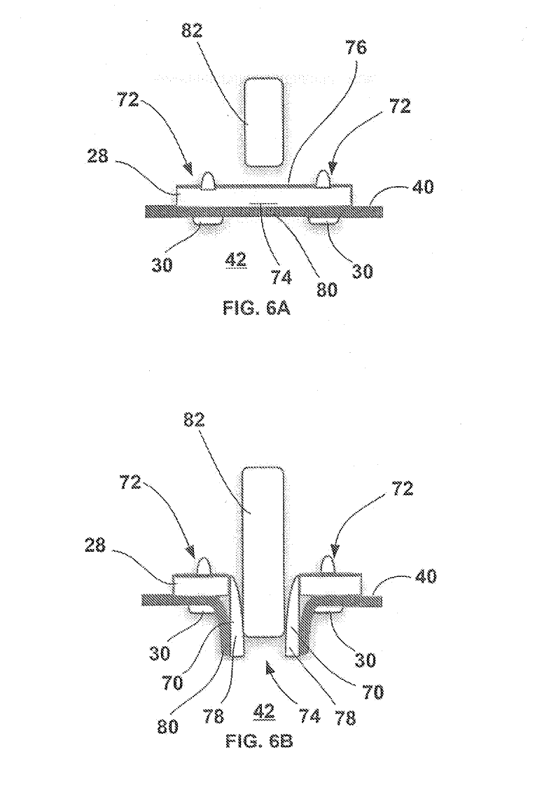

In an example of use FIGS. 6A and 6B illustrate one variation of access port 28 secured via securement mechanisms 30 upon or against vessel wall 40. With retractable flaps 78 created, a catheter 82 may be advanced into opening 74 through access port 28. As catheter 82 is urged through access port 28, as shown in FIG. 6B, the flaps 78 may be urged at least partially into vessel lumen 42 while deforming the frame members 70 extending over or along each flap 78. The edges of the tissue wall opening 80, if also cut into a flap configuration, may also extend at least partially into the vessel lumen 42. Moreover, the flaps 78 and frame members 70 may further serve to protect or shield the edges of tissue opening 80 from directly contacting the catheter 82, which may inhibit or prevent any further trauma to the surrounding tissue.

The catheter 82 may be further advanced into the vessel lumen 42 with the flaps 78 temporarily sealing against the catheter shaft 82. Moreover, during use when an instrument or catheter is passed through the access port 28, the flaps 78 may con form around the instrument or catheter shaft 82 advanced therethrough such that the flaps 78 automatically self-adjust to appropriately position themselves with respect to any instrument passed therethrough. Upon withdrawal of catheter 82 from access port 28, the frame members 70 may bias the flaps 78 back into its un-biased configuration such that each flap 78 re-aligns adjacent to one another and provides hemostasis.

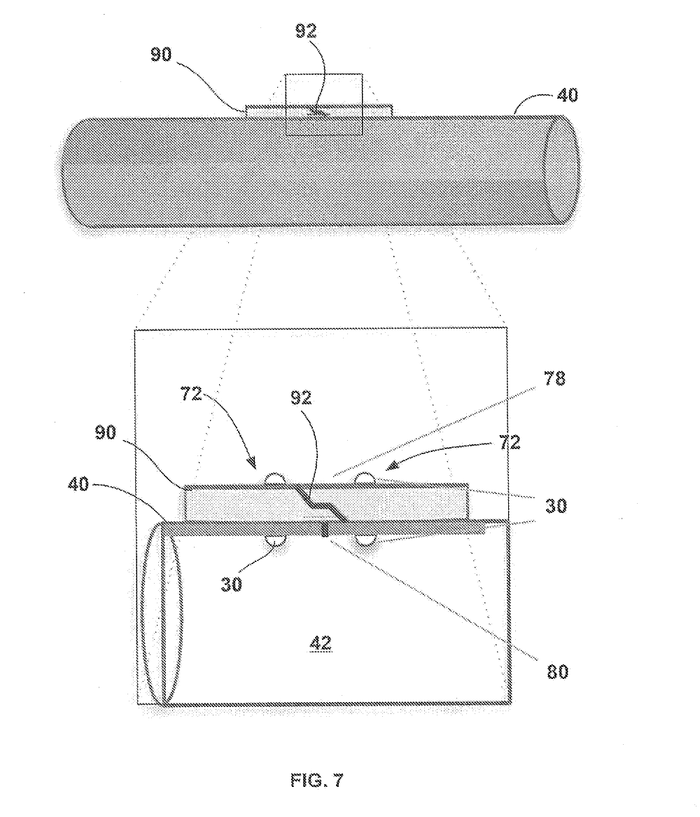

In another variation of the access port, FIG. 7 illustrates an access port 90 having an overlapping flap edge 92 which may be pre-formed in port 90 prior to deployment. In this variation, which shows access port 90 secured upon or against vessel wall 40 via securement mechanisms 30 in the side and detailed view, respectively. Overlapping flap edge 92 may be formed between each individual flap 78 to enhance the hemostatic properties of access port 90 when the flaps 78 are closed with respect to one another.

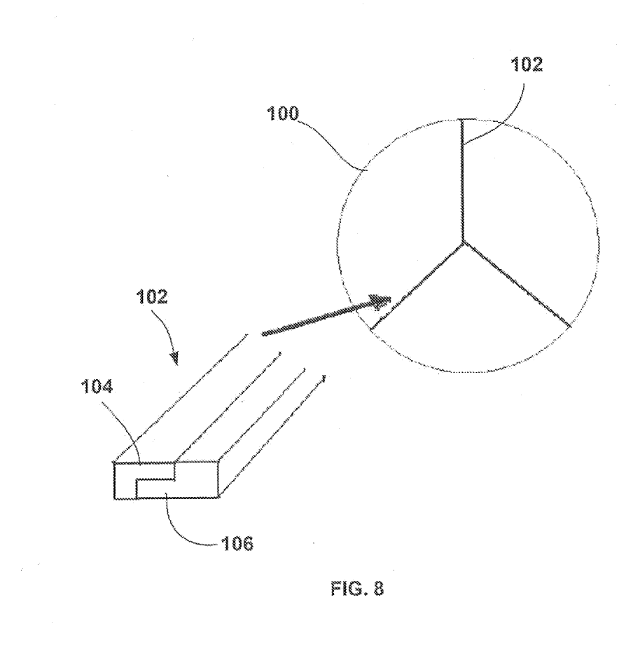

FIG. 8 illustrates yet another variation of an access port 100 having frame or scaffold members 102 which are encapsulated in the surrounding material and which interface with adjacent frame members in an interlocking manner, as shown in the top and detailed perspective views. When all instrument or catheter has been withdrawn from access port 100, the individual flaps may be biased back into their closed port configuration such that adjacent scaffold members 104, 106, for instance, may close adjacent to one another in an overlapping configuration to further enhance the hemostatic properties of port 100.

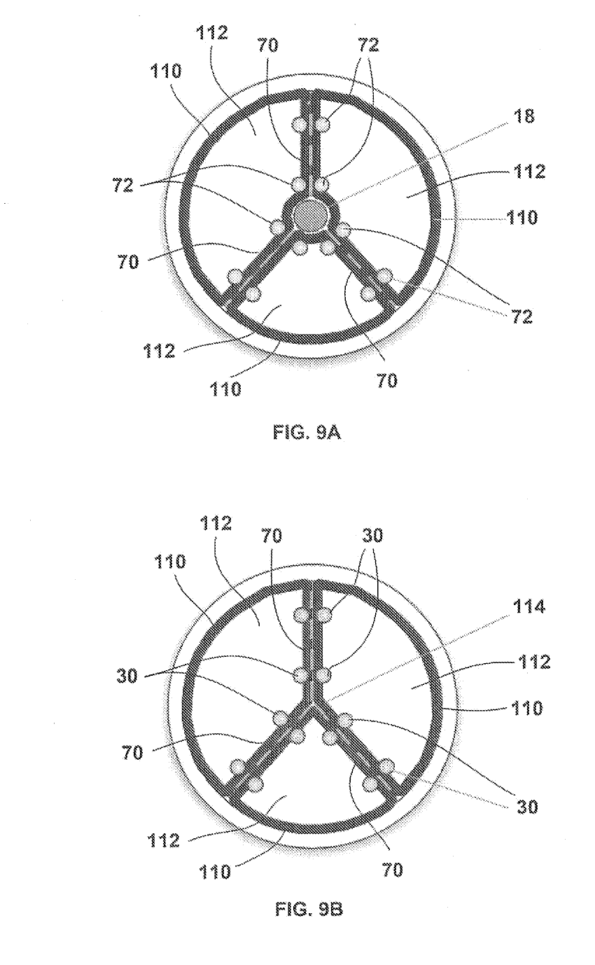

Yet another variation of an access port is illustrated in the top views of FIGS. 9A and 9B. FIG. 9A illustrates an access port having at least three flaps 112 which are each supported by a circularly-shaped outer frame or scaffold 110 along with scaffold members 70. An example of an elongate shaft 18 may be seen passed through the deformed center of the access port and the conformance of each flap 112 around the inserted shaft 18. As shown in FIG. 9B, support mechanisms 30 may be seen securing the access port with the elongate shaft 18 removed and the deformed flaps 112 and scaffold members 70 returned to their unbiased and closed configuration 114.

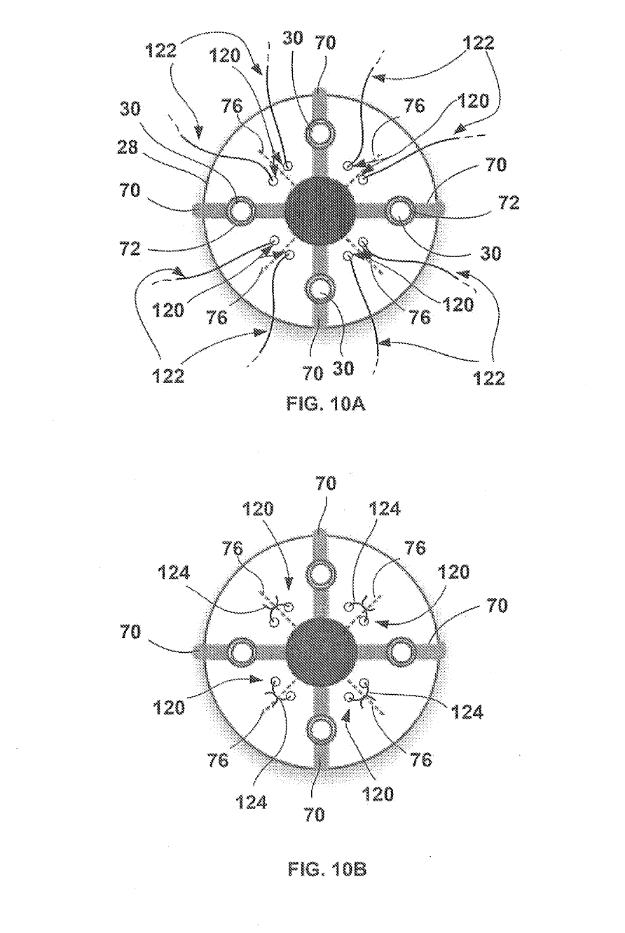

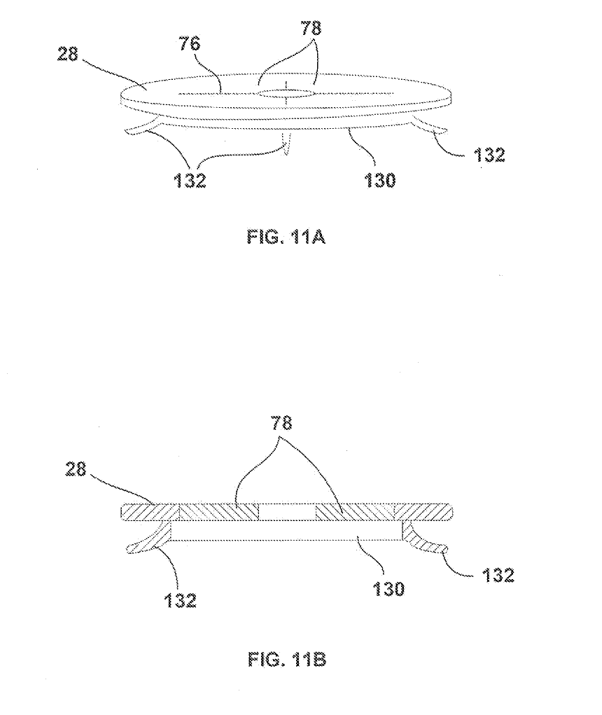

In yet another variation of the access port, FIG. 10A illustrates an access port 28 having suture openings 120 defined through each adjacent flap along opposing sides of each flap cut 76. One or more lengths of suture 122 may be seen passed through each suture opening 120. During deployment and securement of access port 28 as well as during use, the lengths of suture 122 may be left extending from suture openings 120; however, when a procedure is concluded, each of the suture lengths 122 may be optionally tied 124 to one another such that each adjacent flap is secured. Aside from suture, other variations may utilize clips, staples, adhesives, etc., to further secure adjacent flaps to one another.

In yet another variation of an access port 28, FIGS. 11A and 11B illustrate perspective and cross-sectional side views of a variation where access port 28 may incorporate a base 130 with one or more retractable or adjustable members 132 which extend at least partially in a radial manner. Any number of members 132 may be utilized and they may be configured into any number of shapes to provide an atraumatic attachment to the tissue. In use, the port 28 may be placed upon a vessel wall surface with the one or more members 132 extending into the vessel lumen such that the edges of the vessel opening are retained between access port 28 and members 132 in a secure manner. The access port 28 may function in a similar manner as described above.

The applications of the devices and methods discussed above are not limited to controlling access to vessel lumens but may include other body lumens. Modification of the above-described assemblies and methods for carrying out the invention, combinations between different variations as practicable, and variations of aspects of the invention that are obvious to those of skill in the art are intended to be within the scope of the claims.

What is claimed is:

1. A vascular port configured to control entry of an instrument or catheter into a vessel lumen, comprising:
a port sized for securement upon the vessel lumen, the port defining an opening therethrough with one or more flaps;
at least one elastically deformable scaffold member integrated with the port;
wherein the one or more flaps are deformable into a open port configuration when the instrument or catheter is inserted through the opening, and
wherein the at least one member is further biased to reconfigure the one or more flaps back to a closed port configuration upon removal of the instrument or catheter.

2. The vascular port of claim 1 wherein the port has a shape selected from the group consisting of circular disks, ellipses, rectangles, triangles.

3. The vascular port of claim 1 wherein the at least one elastically deformable scaffold member is cross-shaped over the port.

4. The vascular port of claim 1 wherein the at least one elastically deformable scaffold member comprises a frame around a periphery of the one or more flaps.

5. The vascular port of claim 1 wherein the at least one elastically deformable scaffold member is comprised, of a shape memory alloy.

6. The vascular port of claim 5 wherein the shape memory alloy comprises a Nickel-Titanium alloy.

7. The vascular port of claim 1 wherein the one or more flaps are configured into the port when deployed against the vessel lumen.

8. The vascular port of claim 1 wherein the one or more flaps each define an edge which overlaps with an adjacent flap to inhibit leakage of fluids therethrough.

9. The vascular port of claim 1 further comprising one or more lengths of suture which are passable between corresponding openings defined between the one or more flaps.

10. A vascular port configured to conform to an instrument or catheter passed into a vessel, comprising:
a port sized for securement upon the vessel, the port defining an opening therethrough with one or more flaps;
an elastically deformable scaffold integrated with the one or more flaps;
wherein the one or more flaps are configured to self-adjust with respect to the instrument or catheter such that the port conforms thereto, and
wherein the one or more flaps are further configured to form a secure closure upon removal of the instrument or catheter such that hemostasis is maintained through the port.

11. The vascular port of claim 10 wherein the at least one elastically deformable scaffold member comprises a frame around a periphery of the one or more flaps.

12. The vascular port of claim 10 wherein the at least one elastically deformable scaffold member is comprised of a shape memory alloy.

13. The vascular port of claim 12 wherein the shape memory alloy comprises a Nickel-Titanium alloy.

14. The vascular port of claim 10 wherein the one or more flaps each define an edge which overlaps with an adjacent flap to inhibit leakage of fluids therethrough.

15. A vascular port configured to provide access to a vessel through an opening, comprising:
a port configured to be secured upon the vessel, the port defining an opening therethrough with one or more resilient flaps,
wherein the one or more flaps are configured to provide an opening having a diameter greater than 12 F into the vessel for an instrument or catheter, and
wherein the one or more flaps form a secure closure upon removal of the instrument or catheter such that hemostasis is maintained through the port.

16. The vascular port of claim 15 wherein the at least one elastically deformable scaffold member comprises a frame around a periphery of the one or more flaps.

17. The vascular port of claim 15 wherein the at least one elastically deformable scaffold member is comprised of a shape memory alloy.

18. The vascular port of claim 15 wherein the diameter ranges from 12 F to 24 F.

19. The vascular port of claim 15 wherein the one or more flaps each define an edge which overlaps with an adjacent flap to inhibit leakage of fluids therethrough.

20. A vascular port configured to provide access to a vessel through an opening, comprising:
a port defining an opening therethrough with one or more resilient flaps, wherein the one or more flaps form a self-adjusting opening which form a secure closure upon removal of an instrument or catheter from the opening such that hemostasis is maintained through the port; and
one or more adjustable members extending radially from a base of the port, wherein the one or more adjustable members are configured to extend into a vessel lumen when placed upon a vessel wall surface such that the edges of the vessel opening are retained via the one or more members.

21. The vascular port of claim 20 wherein the at least one elastically deformable scaffold member comprises a frame around a periphery of the one or more flaps.

22. The vascular port of claim 20 wherein the at least one elastically deformable scaffold member is comprised of a shape memory alloy.

23. The vascular port of claim 22 wherein the shape memory alloy comprises a Nickel-Titanium alloy.

24. The vascular port of claim 20 wherein the one or more flaps each define an edge which overlaps with an adjacent flap to inhibit leakage of fluids therethrough.

25. A vascular port configured to provide an opening into a vessel for an instrument, comprising:
a port sized for securement upon the vessel, the port defining an opening therethrough with one or more flaps;
an elastically deformable scaffold integrated with the one or more flaps;
wherein the one or more flaps are configured to self-adjust with respect to the instrument or catheter such that the port conforms thereto and further forms a secure closure upon removal of the instrument or catheter such that hemostasis is maintained through the port, and
wherein the port is further configured to facilitate re-entry of the instrument or catheter therethrough.
